(19) United States
(12) Patent Application Publication
(10) Pub. No.: US 2005/0131065 A1
(43) Pub. Date: Jun. 16, 2005

(54) Active substance combination of creatine and/or creatinine and a retinoid

(75) Inventors: Helga Biergiesser, Reinbek (DE); Thomas Blatt, Wedel (DE); Melanie Schmidt, Hamburg (DE); Franz Stab, Echem (DE); Uwe Schonrock, Nahe (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 10/995,203

(22) Filed: Nov. 24, 2004

(30) Foreign Application Priority Data
Nov. 26, 2003 (DE) 103 55 715.6

(51) Int. Cl. A61K 31/198; A61K 31/22; A61K 31/07

(52) U.S. Cl. 514/546; 514/565; 514/725

(57) ABSTRACT

A combination comprising creatine and/or creatinine and/or a derivative thereof and one or more retinoids. This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 103 55 715.6, filed Nov. 26, 2003, the disclosure of which is expressly incorporated by reference herein in its entirety.

### BACKGROUND OF THE INVENTION

1. Field of the Invention

The present invention relates to the use of creatine and/or creatine derivatives and/or creatinine and/or creatinine derivatives in cosmetic or dermatological preparations for the treatment and prophylaxis of the symptoms of UV and/or ozone-induced skin damage and of inflammatory and degenerative skin conditions.

2. Discussion of Background Information

Cosmetic skin care is primarily understood to mean strengthening or restoring the natural function of the skin as a barrier against environmental influences (for example, dirt, chemicals, microorganisms) and against the loss of endogenous substances (for example, water, natural fats, electrolytes).

Impairment of this function may lead to increased resorption of toxic or allergenic substances or to attack by microorganisms, resulting in toxic or allergic skin reactions.

Another aim of skin care is to compensate for the loss by the skin of sebum and water caused by daily washing. This is particularly important if the natural regeneration ability is inadequate. Furthermore, skin care products should protect against environmental influences, in particular against sun and wind, and delay skin aging.

Chronological skin aging is caused, for example, by endogenous, genetically determined factors. The following structural damage and functional disorders can arise, also under the term “senile xerosis”, for example, in the epidermis and the dermis as a result of aging:
a) Dryness, roughness and formation of dryness wrinkles,
b) Itching and altered pore structure of the skin
c) Reduced regreasing by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders arise in the skin in particular as a result of exogenous factors; these are more far-reaching than the degree and quality of the damage in the case of chronological aging:
d) Visible vascular dilation (teleangiectases, cuperosis);
e) Flaccidity and formation of wrinkles;
f) Local hyperpigmentation, hypopigmentation and abnormal pigmentation (e.g., senile keratoses) and
g) Increased susceptibility to mechanical stress (e.g., cracking).

The present invention relates in particular to products for the care of skin that has aged naturally and to the treatment of secondary damage from light-aging, in particular the phenomena listed under a) through g).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development, there are considerable difficulties in stabilizing the active ingredients to an adequate extent against oxidative decay. The use of products containing vitamin A acid, moreover, often causes severe erythematous skin irritations. Retinoids can therefore only be used in low concentrations.

In particular, the present invention relates to cosmetic preparations with an effective protection against harmful oxidation processes in the skin, but also to the protection of cosmetic preparations themselves or to the protection of the constituents of cosmetic preparations against harmful oxidation processes.

The harmful effect of the ultraviolet part of solar radiation on the skin is generally known. Whereas rays with a wavelength of less than about 290 nm (the so-called UVC range) are absorbed by the ozone layer in the earth's atmosphere, rays in the range between about 290 nm and about 320 nm, the so-called UVB range, cause erythema, simple sunburn or even burns of greater or lesser severity.

The narrower range around 308 nm is indicated to be the maximum of the erythematous effect of sunlight.

Numerous compounds are known for protecting against UVB radiation; these are derivatives of 3-benzylidene camphor, of 4-aminobenzoic acid, of cinnamic acid, of salicylic acid, of benzophenone and also of 2-phenylbenzimidazole.

It is also important to have available filter substances for the range between about 320 nm and about 400 nm, the so-called UVA region, since its rays can cause reactions in cases of photosensitive skin. It has been found that UVA radiation leads to damage of the elastic and collagen fibers of connective tissue, which results in premature aging of the skin, and is to be regarded as a cause of numerous phototoxic and photoallergic reactions. The harmful effect of UVB radiation can be intensified by UVA radiation.

To protect against rays of the UVA region, therefore, certain derivatives of dibenzoylmethane are used, the photostability of which is inadequate (Int. J. Cosm. Science 10, 53 (1988), the entire disclosure whereof is expressly incorporated by reference herein).

The UV radiation can, however, also lead to photochemical reactions, in which case the photochemical reaction products again interfere with the skin's metabolism.

Such photochemical reaction products are primarily free-radical compounds, for example hydroxyl radicals. Undefined free-radical photoproducts which form in the skin itself can also result in uncontrolled secondary reactions due to their high reactivity. Furthermore, singlet oxygen, a non-free-radical excited state of the oxygen molecule, can also arise during UV irradiation, as can short-lived epoxides and many others. Singlet oxygen, for example, differs from triplet oxygen (free-radical ground state), which is normally present, by its increased reactivity. However, excited, reactive (free-radical) triplet states of the oxygen molecule also exist.

UV radiation is also a type of ionizing radiation. There is therefore the risk that ionic species will also form during UV exposure, which then for their part are able to interfere oxidatively with the biochemical processes.

In order to prevent these reactions, additional antioxidants and/or free-radical scavengers can be incorporated into the cosmetic or dermatological formulations.

It has already been proposed to use vitamin E, a substance with known antioxidant activity, in sunscreen formulations, although, here too, the effect achieved falls far short of expectations.

It would be advantageous to have available cosmetically, dermatologically and pharmaceutically active substances and preparations, and sunscreen formulations for the prophylaxis and treatment of photosensitive skin, in particular photodermatoses, preferably PLD.

Further terms for polymorphous light dermatosis are PLD, PLE, Mallorca acne and numerous other terms as given in the literature (e.g., A. Voelckel et al., Zentralblatt Haut-und Geschlechtskrankheiten (1989), 156, p. 2, the entire disclosure whereof is expressly incorporated by reference herein).

Antioxidants are mainly used as substances which protect against the deterioration of the preparations in which they are present. Nevertheless, it is known that in human and animal skin undesired oxidation processes can occur as well. Such processes play an important role in skin aging.

The article “Skin Diseases Associated with Oxidative Injury” in “Oxidative Stress in Dermatology”, p. 323 ff. (Marcel Decker Inc., New York, Basel, Hong Kong, Editor: Jürgen Fuchs, Frankfurt, and Lester Packer, Berkeley/Calif.), the entire disclosure whereof is expressly incorporated by reference herein, discusses oxidative skin damage and its more direct causes.

Also for the reason of preventing such reactions, antioxidants and/or free-radical scavengers can be additionally incorporated into cosmetic or dermatological formulations.

A number of antioxidants and free-radical scavengers are known. For example U.S. Pat. Nos. 4,144,325 and 4,248,861, the entire disclosures whereof are expressly incorporated by reference herein, and numerous other documents have already proposed the use of vitamin E, a substance with known antioxidant activity in sunscreen formulations, although here, too, the effect achieved falls far short of the desired effect.

The advantageous prophylactic and therapeutic effect of creatine in cosmetic and medical skin care is known per se. Creatine (from the Greek: τo κρεαç=“the meat”) is characterized by the following structure:

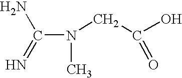

Creatine is found in the myoserum of vertebrates in amounts of 0.05-0.4%, in small amounts also in the brain and blood. As a monohydrate, it is a colorless, crystalline powder. In aqueous solution, creatinine is formed. In the organism, it is formed by the transamidination of L-arginine on glycine to afford guanidinoacetic acid, and subsequent methylation thereof by means of S-adenosyl methionine (by guanidinoacetate methyltransferase). Creatine is regarded as an appetite-promoting constituent of beef and meat extract. The addition of creatine to the diet enhances physical performance.

The prior art is extensive on the cosmetic and dermatological uses of creatine. Thus, DE 100 32 964, the entire disclosure whereof is expressly incorporated by reference herein, describes the use of creatine and/or creatine derivatives in cosmetic or dermatological preparations for the treatment and prophylaxis of the symptoms of UV-induced and/or ozone-induced skin damage and of inflammatory and degenerative skin conditions.

JP 2000/247866, the entire disclosure whereof is expressly incorporated by reference herein, describes skin cosmetics with a content of creatine and/or creatinine which can be used as a cream or as a milky lotion, where excellent skin care properties are attributed to the relevant preparations.

Furthermore, WO 00/33787, the entire disclosure whereof is expressly incorporated by reference herein, describes the use of creatinine as an effective constituent of deodorants.

Moreover, EP-A-565 010, the entire disclosure whereof is expressly incorporated by reference herein, describes hair growth and hair dye preparations with a content of creatinine phosphate.

Finally, U.S. Pat. No. 4,590,067 and EP-A-178 602, the entire disclosures whereof are expressly incorporated by reference herein, describe the use of creatine or creatinine to produce preparations with anti-inflammatory effect.

However, there is the disadvantage that in aqueous products creatine and creatinine crystallize easily, whereby crystals with non-cosmetic impression form and the effectiveness of the product is reduced.

It is desirable to find ways of avoiding the disadvantages of the prior art. In particular, the effect of remedying the damage associated with endogenous, chronological and exogenous skin aging and the prophylaxis should be durable, sustained and without the risk of side effects.

It would further be advantageous to find a form of administering creatine that is characterized by a reduced tendency to form creatine crystals.

### SUMMARY OF THE INVENTION

The present invention provides an active substance combination which comprises
(a) creatine and/or creatinine and/or a creatine derivative and/or a creatinine derivative, and
(b) one or more retinoids.

In one aspect of the combination, the weight ratio of creatinine to creatine may be from about 10:1 to about 1:10, e.g., from about 4:1 to about 3:7, or from about 2:1 to about 1:2.

In another aspect of the combination, the one or more retinoids may comprise retinol and/or retinyl palmitate.

The present invention also provides a cosmetic or dermatological preparation which comprises an effective amount of the active substance combination of the present invention, including the various aspects thereof, as set forth above.

In one aspect, the preparation may comprise from about 0.0005% to about 50% by weight of the active substance combination, e.g., from about 0.01% to about 20% by weight, from about 0.02% to about 10% by weight, from about 0.02% to about 5% by weight, or from about 0.5% to about 3% by weight of the active substance combination, based on the total weight of the preparation.

In another aspect, the preparation may comprise from about 0.001% to about 10% by weight, e.g., from about 0.01% to about 1% by weight of creatine and/or a creatine derivative, based on the total weight of the preparation.

In yet another aspect, the preparation may comprise from about 0.001% to about 10% by weight, e.g., from about 0.01% to about 1% by weight of creatinine and/or a creatinine derivative, based on the total weight of the preparation.

In a still further aspect, the preparation may comprise from about 0.001% to about 10% by weight, e.g., from about 0.01% to about 1% by weight of one or more retinoids, based on the total weight of the preparation.

In yet another aspect, the one or more retinoids may comprise retinol and/or retinyl palmitate.

In another aspect, the preparation may further comprise from about 0.001% to about 30% by weight, e.g., from about 0.01% to about 15% by weight, or from about 1% to about 7% by weight of glycerin, based on the total weight of the preparation.

The present invention also provides a cosmetic or dermatological preparation which comprises from about 0.01% to about 20% by weight of an active substance combination comprising
(a) creatine and/or creatinine and/or a creatine derivative and/or a creatinine derivative, and
(b) one or more retinoids.

In one aspect of the preparation, the weight ratio of creatinine to creatine in the active substance combination may be from about 4:1 to about 3:7, e.g., from about 2:1 to about 1:2.

In another aspect, the preparation may comprise from about 0.02% to about 10% by weight, e.g., from about 0.5% to about 3% by weight, of the active substance combination, based on the total weight of the preparation.

In another aspect, the preparation may comprise from about 0.01% to about 1% by weight of creatine and/or a creatine derivative and/or from about 0.01% to about 1% by weight of creatinine and/or a creatinine derivative, based on the total weight of the preparation.

In yet another aspect, the preparation may further comprise from about 0.01% to about 15% by weight, e.g., from about 1% to about 7% by weight of glycerin, based on the total weight of the preparation.

In a still further aspect, the preparation may comprise from about 0.001% to about 10% by weight, e.g., from about 0.01% to about 1% by weight, of the one or more retinoids.

In another aspect of the preparation, the one or more retinoids may comprise retinol and/or retinyl palmitate.

The present invention also provides an O/W cream, an O/W emulsion, a gel cream and a hydrodispersion, all of which comprise the active substance combination of the present invention, including the various aspects thereof, as set forth above.

The present invention also provides a method for the prophylaxis or treatment of UV or ozone-induced skin damage and a method for the prophylaxis or treatment of inflammatory and degenerative skin. These methods comprise the application to at least a part of the skin of the preparation of the present invention, including the various aspects thereof.

A cosmetic or dermatological preparation according to the invention preferably comprises from about 0.001%, e.g., from about 0.01%, to about 10%, e.g., to about 1% by weight of creatine and/or creatine derivatives, based on the total weight of the preparation.

If derivatives of creatine are used, the preferred derivative is creatine phosphate, which has the following structure:

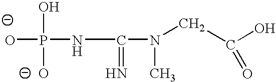

Creatine phosphate is present in fresh muscle where it plays an important role as an energy-storing phosphate (phosphagen). In the working muscle, adenosine 5′-triphosphate (ATP) and creatine are formed from creatine phosphate and adenosine 5′diphosphate under the influence of the enzyme creatine kinase. In the resting muscle the reverse reaction takes place.

Additionally, creatine sulfate, creatine acetate, creatine ascorbate and the derivatives esterified on the carboxyl group with mono- or polyfunctional alcohols are further non-limiting examples of advantageous creatine derivatives for use in the present invention.

Creatinine (likewise from the Greek: τo κρεαç=“the meat”) is characterized by the following structure:

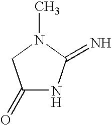

It is formed in the organism through nonenzymatic conversion of creatine phosphate according to the equation: 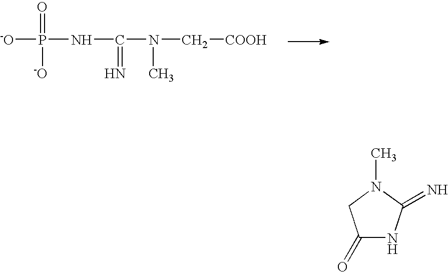
and is secreted through the kidneys. The amount of the creatinine secretion is proportional to the muscle mass and is virtually constant for each individual. Creatinine is contained in meat extract and meat broth cubes.

A cosmetic or dermatological preparation according to the invention preferably comprises from about 0.001%, or from about 0.01% to about 10%, e.g., to about 1% by weight of creatinine and/or creatinine derivatives, based on the total weight of the preparation.

According to the present invention, creatine may advantageously be used without the presence of creatinine, and creatinine may advantageously be used without the presence of creatine. However, it is particularly advantageous to use both substances simultaneously in the active substance combinations and preparations according to the present invention, in particular if the weight ratio of creatinine to creatine is selected from about 10:1 to about 1:10, preferably from about 4:1 to about 3:7, more preferably from about 2:1 to about 1:2.

The group of retinoids which are advantageous according to the invention encompasses, in conceptual terms, all cosmetically and/or pharmaceutically acceptable retinoids, including retinol and its esters, retinal and retinoic acid and esters thereof.

Retinol is characterized by the following structure:

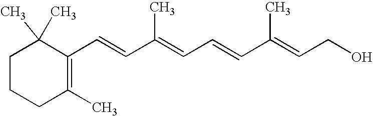

Retinol (also: axerophthol; [3,7-dimethyl-9-(2,6,6-trimethyl-1-cyclohexenyl)-2,4,6,8-nonatetraen-1-ol) is synonymous with vitamin Al and is, analogously to the derivatives retin-1-carboxylic acid (vitamin A acid, retinoic acid, tretinoin) and esters thereof or retin-1-al (vitamin A aldehyde), also sometimes called vitamin A alcohol.

According to the invention, retinol esters may be used equally advantageously, either alone, or with one another or in combination with unesterified retinol in the active substance combinations according to the invention. The retinol esters for use in the invention preferably have the structure 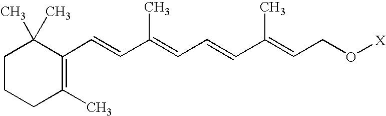
where X is preferably a branched or unbranched alkanoyl or alkenoyl radical having from 1 to about 25 carbon atoms. Retinol palmitate (=retinyl palmitate) is preferably chosen as retinol ester.

Retinal is characterized by the structure:

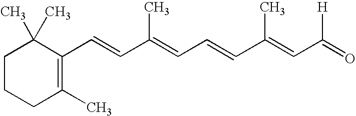

Retinal [vitamin A1 aldehyde, 3,7-dimethyl-9-(2,6,6-trimethyl-1-cyclohexenyl)-2,4,6,8-nonatetraenal] is most stable in its all-trans form. Retinal, which was previously called retinene, forms, bonded to opsins, the sight pigments rhodopsin and iodopsin, as well as bacteriorhodopsin which serves other functions. Retinal forms by the oxidative cleavage of carotene.

Retinoic acid [vitamin A acid, all-trans-3,7-dimethyl-9-(2,6,6-trimethyl-1-cyclohexenyl)-2,4,6,8-nonatetraenoic acid] is characterized by the structure

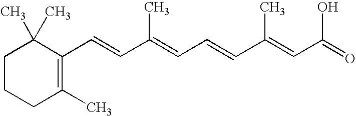

It is effective by suppressing sebum production in cases of acne which are particularly severe, but it also has teratogenic activity. Nevertheless, in certain medically indicated cases, the use of retinoic acid or its esters may be advantageous and is in this regard to be seen in such cases as “acceptable.”

A cosmetic or dermatological preparation according to the invention preferably comprises from about 0.001%, e.g., from about 0.01% to about 10%, e.g., to about 1% by weight of one or more retinoids, in particular retinol, based on the total weight of the preparations.

According to the invention, the active substance combinations are preferably used in cosmetic or dermatological preparations with a content of from about 0.0005% e.g., from about 0.01%, from about 0.02%, or from about 0.5% to about 50%, e.g., to about 20%, to about 10%, to about 5%, or to about 3% by weight, based on the total weight of the preparation.

The effective substance combinations according to the invention can easily be incorporated into established cosmetic or dermatological formulations, advantageously in pump sprays, aerosol sprays, creams, ointments, tinctures, lotions, nail care products (e.g., nail polishes, nail polish remover, nail balms) and the like.

It may also be possible and in some instances advantageous to combine the active substance combinations according to the invention with other active substances, for example with other antimicrobial, antimycotic or antiviral substances.

It is advantageous to buffer the compositions according to the invention. A pH range of from about 3.5 to about 7.5 is advantageous. It is particularly advantageous to select the pH in a range of from about 4.0 to about 6.5.

The cosmetic and/or dermatological preparations according to the invention can have the customary composition and can be used for treating the skin and/or the hair in the sense of a dermatological treatment or a treatment in the sense of care cosmetics. They can however also be used in makeup products in decorative cosmetics.

The cosmetic and/or dermatological formulations of the present invention may have a conventional composition and can be used to treat the skin and/or the hair in terms of a dermatological treatment or a treatment in terms of cosmetic care. However, they can also be used in cosmetic products for decorative cosmetics.

The cosmetic and dermatological preparations which are in the form of a sunscreen are advantageous. They may advantageously additionally comprise at least one UVA filter and/or at least one UVB filter and/or at least one inorganic pigment.

Cosmetic formulations according to the invention for the protection of the skin against UV rays can take various forms, as are, e.g., customarily used for this type of preparation. By way of non-limiting example, they can be present in the form of a solution, an emulsion of the water-in-oil (W/O) type or of the oil-in-water (O/W) type, or a multiple emulsion, for example of the water-in-oil-in-water (W/O/W) type, a gel, a hydrodispersion, a solid stick, or also an aerosol.

The cosmetic formulations according to the invention may comprise cosmetic auxiliaries as are customarily used in such preparations, e.g., preservatives, bactericides, antioxidants, perfumes, antifoams, dyes, coloring pigments, thickeners, surface-active substances, emulsifiers, emollients, moisturizers and/or humectants, fats, oils, waxes and other customary constituents of a cosmetic formulation, such as alcohols, polyols, polymers, foam stabilizers, electrolytes, organic solvents or silicone derivatives.

If the cosmetic or dermatological formulation is present in the form of a solution or lotion, non-limiting examples of solvents which may be used include:
water or aqueous solutions;
oils, such as triglycerides of capric or of caprylic acid, but preferably castor oil;
fats, waxes and other natural and synthetic fatty substances, preferably esters of fatty acids with alcohols of low carbon number, e.g., with isopropanol, propylene glycol or glycerin, or esters of fatty alcohols with alkanoic acids of low carbon number or with fatty acids;
alcohols, diols or polyols of low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the above-mentioned solvents may be used. In the case of alcoholic solvents, water may be a further constituent.

According to the invention, favorable antioxidants which may be used include all antioxidants which are suitable or customary for cosmetic and/or dermatological applications.

The antioxidants are advantageously chosen from the group consisting of amino acids (for example, glycine, histidine, tyrosine, tryptophan) and derivatives thereof, imidazoles (for example, urocanic acid) and derivatives thereof, peptides, such as D,L-carnosine, D-carnosine, L-camosine and derivatives thereof (for example, anserine), carotenoids, carotenes (for example, α-carotene, β-carotene, lycopene) and derivatives thereof, lipoic acid and derivatives thereof (for example, dihydrolipoic acid), aurothioglucose, propylthiouracil and other thiols (for example, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example, buthionine sulfoximines, homocysteine sulfoximine, buthionine sulfones, penta-, hexa- and heptathionine sulfoximine) in very low tolerated doses (for example, pmol to μmol/kg), and furthermore (metal) chelating agents (for example, α-hydroxy-fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA and derivatives thereof, unsaturated fatty acids and derivatives thereof (for example, γ-linolenic acid, linoleic acid, oleic acid), licochalcones, licochalcone A (from radix glycyrrhizae inflatae), folic acid and derivatives thereof, ubiquinone and ubiquinol and derivatives thereof, vitamin C and derivatives (for example, ascorbyl palmitate, Mg ascorbyl phosphate, Na ascorbyl phosphate, ascorbyl acetate), tocopherols and derivatives (for example, vitamin E acetate), vitamin A and derivatives (vitamin A palmitate) and coniferyl benzoate of benzoin resin, rutic acid and derivatives thereof, isoflavones (isoflavone 150) and isoflavonoids, ferulic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example, selenium methionine), stilbenes and derivatives thereof (for example, stilbene oxide, trans-stilbene oxide) and the derivatives of these active substances mentioned which are suitable according to the invention (salts, esters, ethers, sugars, nucleotides, nucleosides, peptides and lipids).

The amount of the antioxidants (one or more compounds) in the preparations is preferably from about 0.001% to about 30% by weight, particularly preferably from about 0.05% to about 20% by weight, in particular from about 1% to about 10% by weight, based on the total weight of the preparation.

The preparations according to the invention may contain cosmetic auxiliaries, as are customarily used in such preparations, e.g., preservatives, bactericides, deodorants, antiperspirants, insect repellents, vitamins, antifoams, dyes, coloring pigments, thickeners, emollients, moisturizers and/or humectants, fats, oils, waxes and other customary constituents of a cosmetic formulation, such as alcohols, polyols, polymers, foam stabilizers, electrolytes, organic solvents or silicone derivatives.

Moreover, preparations according to the invention may advantageously comprise substances which absorb UV radiation in the UVB range, the total amount of filter substances being, for example, about 0.1% by weight to about 30% by weight, preferably about 0.5 to about 10% by weight, in particular about 1 to about 6% by weight, based on the total weight of the preparations, in order to provide cosmetic preparations that can protect the hair or the skin from the entire range of ultraviolet radiation. They can also be used as sunscreen for the hair.

If the preparations according to the invention contain UVB filter substances, these can be oil-soluble or water-soluble. Non-limiting examples of advantageous oil-soluble filters for use in the invention include, e.g.:
3-benzylidenecamphor und derivatives thereof, preferably 3-(4-methylbenzylidene)-camphor,
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate;
2,4,6-trianilino-(p-carbo-2′-ethyl-1′-hexyloxy)-1,3,5-triazine.

Non-limiting examples of advantageous water-soluble substances include, e.g.:
salts of 2-phenylbenzimidazole-5-sulfonic acid such as, e.g., the sodium, potassium and triethanolammonium salts, and the sulfonic acid itself;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3bornylidenemethyl)sulfonic acid and its salts, and 1,4-di(2-oxo-10-sulfo-3-bornylidenemethyl)-benzene and its salts (the corresponding 10-sulfato compounds, e.g., the corresponding sodium, potassium or triethanolammonium salt) also known as benzene-1,4-di(2-oxo-3-bornylidenemethyl-10-sulfonic acid).

The list of said UVB filters which can be used in combination with the active substance combinations according to the invention is of course not intended to be limiting.

It may also be advantageous to use UVA filters which are customarily present in cosmetic and/or dermatological preparations. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. They may be used in the same amounts that have been given for UVB combinations.

Particularly if crystalline or microcrystalline solids, e.g., inorganic micropigments, are to be incorporated in the preparations according to the invention the latter may also contain anionic, nonionic and/or amphoteric surfactants. Surfactants are amphiphilic substances that can dissolve organic, nonpolar substances in water.

The hydrophilic moieties of a surfactant molecule are mostly polar functional groups, for example —COO⁻, —OSO₃⁻, —SO₃⁻, while the hydrophobic moieties are usually nonpolar hydrocarbon radicals. Surfactants are generally classified according to the type and charge of the hydrophilic molecular moiety. In this connection, it is possible to distinguish between four groups:
anionic surfactants,
cationic surfactants,
amphoteric surfactants and
nonionic surfactants.

Anionic surfactants usually have as functional groups carboxylate, sulfate or sulfonate groups. In aqueous solution, they form negatively charged organic ions in acidic or neutral media. Cationic surfactants are characterized almost exclusively by the presence of a quaternary ammonium group. In aqueous solution they form positively charged organic ions in acidic or neutral media. Amphoteric surfactants contain both anionic and cationic groups and accordingly in aqueous solution behave like anionic or cationic surfactants, depending on the pH value. In strongly acidic media they have a positive charge, and in alkaline media they carry a negative charge. By contrast, in the neutral pH range, they are zwitterionic, as the example below serves to illustrate:

| | | |
|---|---|---|
| RNH₂⁺CH₂CH₂COOHX⁻ | (at pH = 2) | X⁻ = any anion, e.g. Cl⁻ |
| RNH₂⁺CH₂CH₂COO⁻ | (at pH = 7) | |
| RNHCH₂CH₂COO⁻B⁺ | (at pH = 12) | B⁺ = any cation, e.g. Na.⁺ |

Polyether chains are typical of nonionic surfactants. Nonionic surfactants do not form ions in an aqueous medium.

A. Anionic Surfactants

Non-limiting examples of anionic surfactants which may be used advantageously include: Acylamino acids (and salts thereof), such as

1. acyl glutamates, for example, sodium acyl glutamate, di-TEA-palmitoyl aspartate and sodium caprylic/capric glutamate,
2. acylpeptides, for example, palmitoyl-hydrolysed milk protein, sodium cocoyl-hydrolysed soy protein and sodium/potassium cocoyl-hydrolysed collagen,
3. sarcosinates, for example, myristoyl sarcosine, TEA-lauroyl sarcosinate, sodium lauroyl sarcosinate and sodium cocoyl sarcosinate,
4. taurates, for example sodium lauroyl taurate and sodium methylcocoyl taurate,
5. acyl lactylates, lauroyl lactylate, caproyl lactylate
6. alaninates

Carboxylic acids and derivatives thereof, such as
1. carboxylic acids, for example, lauric acid, aluminum stearate, magnesium alkanolate and zinc undecylenate,
2. ester carboxylic acids, for example, calcium stearoyl lactylate, laureth-6 citrate and sodium PEG-4 lauramide carboxylate,
3. ether carboxylic acids, for example sodium laureth-13 carboxylate and sodium PEG-6 cocamide carboxylate.
   Phosphoric esters and salts, such as, for example, DEA-oleth-10 phosphate and dilaureth-4 phosphate,
   Sulfonic acids and salts, such as
1. acyl isethionates, e.g., sodium/ammoniumcocoyl isethionate,
2. alkylarylsulfonates,
3. alkylsulfonates, for example, sodium cocomonoglyceride sulfate, sodium C₁₂₋₁₄-olefin sulfonate, sodium lauryl sulfoacetate and magnesium PEG-3 cocamide sulfate,
4. sulfosuccinate's, for example dioctyl sodium sulfosuccinate, disodium laureth sulfosuccinate, disodium lauryl sulfosuccinate and disodium undecyleneamido-MEA sulfosuccinate, and
Sulfuric esters, such as
1. alkyl ether sulfate, for example sodium, ammonium, magnesium, MIPA, TIPA laureth sulfate, sodium myreth sulfate and sodium C₁₂₋₁₃ pareth sulfate,
2. alkyl sulfates, for example sodium, ammonium and TEA lauryl sulfate.

B. Cationic Surfactants

Non-limiting examples of cationic surfactants which may be advantageously used include

1. alkylamines,
2. alkylimidazoles,
3. ethoxylated amines and
4. quaternary surfactants,
5. ester quats.

Quaternary surfactants contain at least one N atom which is covalently bonded to 4 alkyl or aryl groups. Irrespective of the pH value, this results in a positive charge. Alkylbetaine, alkylamidopropylbetaine and alkylamidopropylhydroxysulfaine are advantageous. The cationic surfactants which may be used according to the invention can also preferably be chosen from quaternary ammonium compounds, in particular benzyltrialkylammonium chlorides or bromides, such as, for example, benzyldimethylstearylammonium chloride, and also alkyltrialkylammonium salts, for example, cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example, lauryl- or cetylpyridinium chloride, imidazoline derivatives and compounds having cationic character, such as amine oxides, for example, alkyldimethylamine oxides or alkylaminoethyldimethylamine oxides. In particular the use of cetyltrimethylammonium salts is advantageous.

C. Amphoteric Surfactants

Non-limiting examples of amphoteric surfactants which may be used advantageously include

1. acyl/dialkylethylenediamine, for example, sodium acyl amphoacetate, disodium acyl amphodipropionate, disodium alkyl amphodiacetate, sodium acyl amphohydroxypropylsufonate, disodium acyl amphodiacetate and sodium acyl amphopropionate,
2. N-alkylamino acids, for example, aminopropylalkylglutamide, alkylaminopropionic acid, sodium alkylimidodipropionate and lauroamphocarboxyglycinate.

D. Nonionic Surfactants

Non-limiting examples of nonionic surfactants which can be used advantageously are

1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerin, sorbitol or other alcohols,
5. ethers, for example ethoxylated/propoxylated alcohols, ethoxylated/propoxylated esters, ethoxylated/propoxylated glycerin esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside.
6. sucrose esters, sucrose ethers
7. polyglycerol esters, diglycerol esters, monoglycerol esters
8. methylglucose esters, esters of hydroxy acids

Also advantageous is the use of a combination of anionic and/or amphoteric surfactants with one or more nonionic surfactants.

The surface-active substance may be present in the preparations according to the invention in a concentration of, for example, from about 1% to about 95% by weight, based on the total weight of the preparation.

The lipid phase of the cosmetic or dermatological emulsions according to the present invention may advantageously be chosen from the following substances:
mineral oils, mineral waxes;
oils, such as triglycerides of capric or of caprylic acid, and also natural oils, such as, for example, castor oil;
fats, waxes and other natural and synthetic fatty substances, preferably esters of fatty acids with alcohols of low carbon number, e.g. with isopropanol, propylene glycol or glycerin, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes, and mixed forms thereof.

The oil phase of the emulsions of the present invention may advantageously be chosen from esters of saturated and/or unsaturated, branched and/or unbranched alkanecarboxylic acids having a chain length of from about 3 to about 30 carbon atoms and saturated and/or unsaturated, branched and/or unbranched alcohols having a chain length of from about 3 to about 30 carbon atoms and from esters of aromatic carboxylic acids and saturated and/or unsaturated, branched and/or unbranched alcohols having a chain length of from about 3 to about 30 carbon atoms. Such ester oils can advantageously be chosen from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g., jojoba oil.

The oil phase may also advantageously be chosen from branched and unbranched hydrocarbons and hydrocarbon waxes, silicon oils, dialkyl ethers, saturated or unsaturated, branched or unbranched alcohols, and fatty acid triglycerides, namely the triglycerol esters of saturated and/or unsaturated, branched and/or unbranched alkanecarboxylic acids having a chain length of from about 8 to about 24 carbon atoms, in particular from about 12 to about 18 carbon atoms. The fatty acid triglycerides can, for example, be advantageously chosen from synthetic, semisynthetic and natural oils, e.g., olive oil, sunflower oil, soy oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase may advantageously be chosen from 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, and dicaprylyl ether.

Particularly advantageous mixtures are those of C₁₂₋₁₅-alkyl benzoate and 2-ethylhexyl isostearate, those of C₁₂₋₁₅-alkyl benzoate and isotridecyl isononanoate, and those of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Advantageously, the oil phase may also have a content of cyclic or linear silicone oils, or consist entirely of such oils, although it is preferred to use an additional content of other oil phase components apart from the silicone oil or the silicone oils.

Cyclomethicone (e.g., decamethylcyclopentasiloxane) may advantageously be chosen as the silicone oil for use in the present invention. However, other silicone oils can also be used advantageously for the purposes of the present invention, for example undecamethylcyclotrisiloxane, polydimethylsiloxane, and poly(methylphenylsiloxane).

Mixtures of cyclomethicone and isotridecyl isononanoate, and of cyclomethicone and 2-ethylhexyl isostearate are also particularly advantageous.

Optionally, the aqueous phase of the preparations according to the invention advantageously comprises alcohols, diols or polyols of low carbon number, and ethers thereof, preferably ethanol, isopropanol, propylene glycol, glycerin, ethylene glycol, ethylene glycol monoethyl or monobutyl ether, propylene glycol monomethyl., monoethyl or monobutyl ether, diethylene glycol monomethyl or monoethyl ether and analogous products, and also alcohols of low carbon number, e.g., ethanol, isopropanol, 1,2-propanediol, glycerin and, in particular, one or more thickeners which can advantageously be chosen from silicon dioxide, aluminum silicates, polysaccharides and derivatives thereof, e.g. hyaluronic acid, xanthan gum, hydroxypropylmethylcellulose, particularly advantageously from the polyacrylates, preferably a polyacrylate from the group of the so-called Carbopols, for example Carbopol grades 980, 981, 1382, 2984, 5984, in each case individually or in combination.

Preparations according to the invention which are present in the form of emulsions contain one or more emulsifiers. O/W emulsifiers may advantageously be chosen, for example, from the group of polyethoxylated, polypropoxylated or polyethoxylated and polypropoxylated products, e.g.:
fatty alcohol ethoxylates,
ethoxylated wool wax alcohols,
polyethylene glycol ethers of the general formula R—O—(—CH₂—CH₂—O—)ₙ—R′,
fatty acid ethoxylates of the general formula R—COO—(—CH₂—CH₂—O—)ₙ—H,
etherified fatty acid ethoxylates of the general formula R—COO—(—CH₂—CH₂—O—)ₙ—R′,
esterified fatty acid ethoxylates of the general formula R—COO—(—CH₂—CH₂—O—)ₙ—C(O)—R′,
polyethylene glycol glycerol fatty acid esters, ethoxylated sorbitan esters,
cholesterol ethoxylates,
ethoxylated triglycerides,
alkyl ether carboxylic acids of the general formula R—O—(—CH₂—CH₂—O—)ₙ—CH₂—COOH wherein n is a number of from about 5 to about 30,
polyoxyethylene sorbitol fatty acid esters, alkyl ether sulfates of the general formula R—O—(—CH₂—CH₂—O—)ₙ—SO₃—H,
fatty alcohol propoxylates of the general formula R—O—(—CH₂—CH(CH₃)—O—)ₙ—H,
polypropylene glycol ethers of the general formula R—O—(—CH₂—CH(CH₃)—O—)ₙ—R′,
propoxylated wool wax alcohols,
etherified fatty acid propoxylates R—COO—(—CH₂—CH(CH₃)—O—)ₙ—R′,
esterified fatty acid propoxylates of the general formula R—COO—(—CH₂—CH(CH₃)—O—)ₙ—C(O)—R′,
fatty acid propoxylates of the general formula R—COO—(—CH₂—CH(CH₃)—O—)ₙ—H,
polypropylene glycol glycerol fatty acid esters,
propoxylated sorbitan esters,
cholesterol propoxylates,
propoxylated triglycerides,
alkyl ether carboxylic acids of the general formula R—O—(—CH₂—CH(CH₃)O—)ₙ—CH₂—COOH,
alkyl ether sulfates or the parent acids of these sulfates of the general formula R—O—(—CH₂—CH(CH₃)—O—)ₙ—SO₃—H,
fatty alcohol ethoxylates/propoxylates of the general formula R—O—Xₙ—Yₘ—H,
polypropylene glycol ethers of the general formula R—O—Xₙ—Yₘ—R′,
etherified fatty acid propoxylates of the general formula R—COO—Xₙ—Yₘ—R′,
fatty acid ethoxylates/propoxylates of the general formula R—COO—Xₙ—Yₘ—H.

According to the invention, the polyethoxylated or polypropoxylated or polyethoxylated and polypropoxylated O/W emulsifiers used are particularly advantageously chosen from the group of substances having HLB values of from about 11 to about 18, very particularly advantageously having HLB values of from about 14.5 to about 15.5, provided the O/W emulsifiers have saturated radicals R and R′. If the O/W emulsifiers have unsaturated radicals R and/or R′, or isoalkyl derivatives are present, the preferred HLB values of such emulsifiers may also be lower or higher.

It is advantageous to choose the fatty alcohol ethoxylates from ethoxylated stearyl alcohols, cetyl alcohols, cetylstearyl alcohols (cetearyl alcohols). Particular preference is given to:
polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20),
polyethylene glycol(12) isostearyl ether (isosteareth-12), polyethylene glycol (13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20),
polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20),
polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether. (isoceteth-18), polyethylene glycol(19) isocetyl ether (isoceteth-19), polyethylene glycol(20) isocetyl ether (isoceteth-20),
polyethylene glycol(12)oleyl ether (oleth-12), polyethylene glycol(13)oleyl ether (oleth-13), polyethylene glycol(14)oleyl ether (oleth-14), polyethylene glycol(15)oleyl ether (oleth-15),
polyethylene glycol(12)lauryl ether (laureth-12), polyethylene glycol(12)isolauryl ether (isolaureth-12),
polyethylene glycol(13)cetylstearyl ether (ceteareth-13), polyethylene glycol(14)cetylstearyl ether (ceteareth-14), polyethylene glycol(15)cetylstearyl ether (ceteareth-15), polyethylene glycol(16)cetylstearyl ether (ceteareth-16), polyethylene glycol(17)cetylstearyl ether (ceteareth-17), polyethylene glycol(18)cetylstearyl ether (ceteareth-18), polyethylene glycol(19)cetylstearyl ether (ceteareth-19), polyethylene glycol(20)cetylstearyl ether (ceteareth-20).

It may also be advantageous to choose the fatty acid ethoxylates from the following:
polyethylene glycol(20)stearate, polyethylene glycol(21)stearate, polyethylene glycol(22)stearate, polyethylene glycol(23)stearate, polyethylene glycol(24)stearate, polyethylene glycol(25)stearate,
polyethylene glycol(12)isostearate, polyethylene glycol(13)isostearate, polyethylene glycol(14)isostearate, polyethylene glycol(15)isostearate, polyethylene glycol(16)isostearate, polyethylene glycol(17)isostearate, polyethylene glycol(18)isostearate, polyethylene glycol(19)isostearate, polyethylene glycol(20)isostearate, polyethylene glycol(21)isostearate, polyethylene glycol(22)isostearate, polyethylene glycol(23)isostearate, polyethylene glycol(24)isostearate, polyethylene glycol(25)isostearate,
polyethylene glycol(12)oleate, polyethylene glycol(13)oleate, polyethylene glycol(14)oleate, polyethylene glycol(15)oleate, polyethylene glycol(16)oleate, polyethylene glycol(17)oleate, polyethylene glycol(18)oleate, polyethylene glycol(19)oleate, polyethylene glycol(20)oleate.

Sodium laureth-11 carboxylate may advantageously be used as the ethoxylated alkyl ether carboxylic acid or salt thereof.

Sodium laureth-1-4 sulfate may advantageously be used as alkyl ether sulfate.

Polyethylene glycol(30) cholesteryl ether may advantageously be used as ethoxylated cholesterol derivative. Polyethylene glycol(25)soyasterol has also proven beneficial.

The polyethylene glycol(60) evening primrose glycerides can advantageously be used as ethoxylated triglycerides.

It may also be advantageous to choose the polyethylene glycol glycerin fatty acid esters from polyethylene glycol(20)glyceryl laurate, polyethylene glycol(21)glyceryl laurate, polyethylene glycol(22)glyceryl laurate, polyethylene glycol(23)glyceryl laurate, polyethylene glycol(6)glyceryl caprate/caprinate, polyethylene glycol(20)glyceryl oleate, polyethylene glycol(20)glyceryl isostearate, polyethylene glycol(18)glyceryl oleate/cocoate.

It may likewise be favorable to choose the sorbitan esters from polyethylene glycol(20)sorbitan monolaurate, polyethylene glycol(20)sorbitan monostearate, polyethylene glycol(20)sorbitan monoisostearate, polyethylene glycol(20)sorbitan monopalmitate, polyethylene glycol(20)sorbitan monooleate.

Non-limiting examples of advantageous W/O emulsifiers which may be used include: fatty alcohols having from about 8 to about 30 carbon atoms, monoglycerol esters of saturated and/or unsaturated, branched and/or unbranched alkanecarboxylic acids having a chain length of from about 8 to about 24, in particular from about 12 to about 18 carbon atoms, diglycerol esters of saturated and/or unsaturated, branched and/or unbranched alkanecarboxylic acids having a chain length of from about 8 to about 24, in particular from about 12 to about 18 carbon atoms, monoglycerol ethers of saturated and/or unsaturated, branched and/or unbranched alcohols having a chain length of from about 8 to about 24, in particular from about 12 to about 18 carbon atoms, diglycerol ethers of saturated and/or unsaturated, branched and/or unbranched alcohols having a chain length of from about 8 to about 24, in particular from about 12 to about 18 carbon atoms, propylene glycol esters of saturated and/or unsaturated, branched and/or unbranched alkanecarboxylic acids having a chain length of from about 8 to about 24, in particular from about 12 to about 18 carbon atoms, and sorbitan esters of saturated and/or unsaturated, branched and/or unbranched alkanecarboxylic acids having a chain length of from about 8 to about 24, in particular from about 12 to about 18 carbon atoms.

Particularly advantageous W/O emulsifiers comprise glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2)stearyl ether (steareth-2), glyceryl monolaurate, glyceryl monocaprinate, glyceryl monocaprylate.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice. Unless stated otherwise, all amounts, fractions and percentages given are based on the weight and the total amount or on the total weight of the preparations.

Examples O/W Creams

| | % by weight |
|---|---|
| Example No. 1 | |
| Glyceryl stearate self-emulsifying | 4.00 |
| PEG-40-stearate | 1.00 |
| Cetyl alcohol | 3.00 |
| Caprylic acid/capric acid triglycerides | 5.00 |
| Paraffinum liquidum | 5.00 |
| Tocopherol | 0.1 |
| Na₃HEDTA | 0.1 |
| Creatine | 1.50 |
| Creatinine | 0.50 |
| Retinyl palmitate | 0.03 |
| Preservatives, perfume | q.s. |
| Polyacrylic acid | 3.00 |
| Aqueous sodium hydroxide 45% | q.s. |
| Glycerin | 5.00 |
| Water | ad 100 |
| Example No. 2 | |
| Glyceryl stearate self-emulsifying | 3.00 |
| Stearic acid | 1.00 |
| Cetyl alcohol | 2.00 |
| Caprylic acid/capric acid triglycerides | 3.00 |
| Dicaprylyl ether | 4.00 |
| Paraffinum liquidum | 2.00 |
| Creatine | 0.30 |
| Creatinine | 0.10 |
| Retinyl palmitate | 0.30 |
| Preservatives, perfume | q.s. |
| Polyacrylic acid | 0.1 |
| Aqueous sodium hydroxide 45% | q.s. |
| Glycerin | 3.00 |
| Butylene glycol | 3.00 |
| Water | ad 100 |
| Example No. 3 | |
| Glyceryl stearate citrate | 2.00 |
| Stearyl alcohol | 2.00 |
| Lanolin alcohol | 1.00 |
| Caprylic acid/capric acid triglycerides | 4.00 |
| Paraffinum liquidum | 8.00 |
| Dimethicone | 1.00 |
| Creatine | 1.00 |
| Creatinine | 0.30 |
| Retinyl palmitate | 0.03 |
| Preservatives, perfume | q.s. |
| Caustic soda solution 45% | q.s. |
| Glycerin | 7.50 |
| Water | ad 100 |
| Example No. 4 | |
| Glyceryl stearate citrate | 2.00 |
| Stearyl alcohol | 2.00 |
| Lanolin alcohol | 1.00 |
| Caprylic acid/capric acid triglycerides | 4.00 |
| Paraffinum liquidum | 8.00 |
| Dimethicone | 1.00 |
| Creatine | 1.00 |
| Creatinine | 1.00 |
| Retinyl palmitate | 0.8 |
| Preservatives, perfume | q.s. |
| Aqueous sodium hydroxide 45% | q.s. |
| Glycerin | 7.50 |
| Dihydroxyacetone | 1.00 |
| Water | ad 100 |
| Example No. 5 | |
| Polyglyceryl-3-methylglucose-distearate | 3.00 |
| Cetyl alcohol | 3.00 |
| Caprylic acid/capric acid triglycerides | 3.00 |
| Dicaprylyl ether | 2.00 |
| Paraffinum liquidum | 3.00 |
| Creatine | 2.00 |
| Creatinine | 0.50 |
| Retinyl palmitate | 0.05 |
| Na₃HEDTA | 0.1 |
| Preservatives, perfume | q.s. |
| Polyacrylic acid | 0.1 |
| Aqueous sodium hydroxide 45% | q.s. |
| Glycerin | 3.00 |
| Water | ad 100 |
| Example No. 6 | |
| Glyceryl stearate citrate | 2.00 |
| Sorbitan stearate | 2.00 |
| Cetyl stearyl alcohol | 2.00 |
| Caprylic acid/capric acid triglycerides | 3.00 |
| Octyldodecanol | 2.00 |
| Dicaprylyl ether | 1.00 |
| Creatine | 1.50 |
| Creatinine | 0.45 |
| Retinyl palmitate | 0.30 |
| Tocopherol | 0.20 |
| Preservatives, perfume | q.s. |
| Polyacrylic acid | 0.1 |
| Aqueous sodium hydroxide 45% | q.s. |
| Glycerin | 3.00 |
| Water | ad 100 |
| Example No. 7 | |
| Glyceryl stearate self-emulsifying | 5.00 |
| Stearyl alcohol | 2.00 |
| Caprylic acid/capric acid triglycerides | 2.00 |
| Octyldodecanol | 2.00 |
| Dimethicone polydimethylsiloxane | 2.00 |
| Titanium dioxide | 2.00 |
| 4-Methylbenzylidene camphor | 1.00 |
| Butylmethoxy dibenzoylmethane | 0.50 |
| Creatine | 1.50 |
| Creatinine | 0.45 |
| Retinyl palmitate | 0.30 |
| Preservatives, perfume | q.s. |
| Polyacrylic acid | 0.15 |
| Aqueous sodium hydroxide 45% | q.s. |
| Glycerin | 3.00 |
| Water | ad 100 |
| Example No. 8 | |
| Glyceryl stearate citrate | 2.00 |
| Cetyl stearyl alcohol | 3.00 |
| C₁₂₋₁₅ Alkylbenzoate | 2.00 |
| Octyldodecanol | 2.00 |
| Paraffinum liquidum | 4.00 |
| Creatine | 5.00 |
| Creatinine | 1.50 |
| Retinyl palmitate | 1.00 |
| 2,4-Bis-(4-(2-ethylhexyloxy-2-hydroxy)-phenyl-6-(4- | 1.0 |
| methoxyphenyl)-(1,3,5)-triazine | |
| Dihydroxyacetone | 0.5 |
| Preservatives, perfume | q.s. |
| Polyacrylic acid | 0.1 |
| Aqueous sodium hydroxide 45% | q.s. |
| Butylene glycol | 3.00 |
| Ethanol | 3.00 |
| Water | ad 100 |
| Example No. 9 | |
| Glyceryl stearate citrate | 2.00 |
| Cetyl stearyl alcohol | 1.00 |
| C₁₂₋₁₅ Alkylbenzoate | 3.00 |
| Paraffinum liquidum | 2.00 |
| Creatine | 1.00 |
| Creatinine | 0.50 |
| Retinyl palmitate | 0.02 |
| 2,4-Bis-(4-(2-ethylhexyloxy-2-hydroxy)-phenyl-6-(4- | 3.0 |
| methoxyphenyl)-(1,3,5)-triazine | |
| Ethylenediamine tetraacetic acid trisodium | 0.20 |
| Preservatives, perfume | q.s. |
| Xanthan gum | 0.20 |
| Aqueous sodium hydroxide 45% | q.s. |
| Glycerin | 3.00 |
| Water | ad 100 |
| Example No. 10 | |
| Stearic acid | 2.50 |
| Cetyl alcohol | 3.00 |
| Octyldodecanol | 4.00 |
| Cyclic dimethylpolysiloxane | 0.50 |
| Creatine | 1.00 |
| Creatinine | 0.50 |
| Retinyl palmitate | 0.02 |
| Preservatives, perfume | q.s. |
| Polyacrylic acid | 0.05 |
| Aqueous sodium hydroxide 45% | q.s. |
| Glycerin | 5.00 |
| Ethanol | 3.00 |
| Water | ad 100 |
| Example No. 11 | |
| Stearic acid | 3.50 |
| Cetyl alcohol | 4.50 |
| Cetyl stearyl alcohol | 0.50 |
| Octyldodecanol | 6.0 |
| Cyclic dimethylpolysiloxane | 2.00 |
| 4-Methylbenzylidene camphor | 1.00 |
| Butylmethoxy dibenzoylmethane | 0.50 |
| Creatine | 2.00 |
| Creatinine | 0.90 |
| Retinyl palmitate | 0.70 |
| 2,4-Bis-(4-(2-ethylhexyloxy-2-hydroxy)-phenyl-6-(4- | 0.5 |
| methoxyphenyl)-(1,3,5)-triazine | |
| Dihydroxyacetone | 0.5 |
| Tocopherol | 0.05 |
| Ethylenediaminetetraacetic acid trisodium | 0.20 |
| Preservatives, perfume | q.s. |
| Polyacrylic acid | 0.05 |
| Aqueous sodium hydroxide 45% | q.s. |
| Glycerin | 3.00 |
| Water | ad 100 |

Examples W/O Emulsions

| | % by weight |
|---|---|
| Example No. 12 | |
| Polyglyceryl-2-dipolyhydroxystearate | 5.00 |
| 2,4-Bis-(4-(2-ethylhexyloxy-2-hydroxy)-phenyl-6-(4- | 2.00 |
| methoxyphenyl)-(1,3,5)-triazine | |
| Diethylhexyl butamidotriazone | 3.00 |
| Octocrylene | 7.00 |
| Diethylhexyl butamidotriazone | 1.00 |
| Phenylene-1,4-bis-(monosodium,-2-benzimidazyl-5,7- | 1.00 |
| disulfonic acid) | |
| Phenylbenzimidazole sulfonic acid | 0.50 |
| Zinc oxide | 3.00 |
| Dicaprylyl ether | 10.00 |
| Dicaprylyl carbonate | 5.00 |
| Phenylmethylpolysiloxane | 2.00 |
| PVP hexadecene copolymer | 0.50 |
| Glycerin | 3.00 |
| Magnesium sulfate | 1.00 |
| Tocopherol acetate | 0.50 |
| Creatine | 0.50 |
| Creatinine | 0.20 |
| Retinyl palmitate | 0.03 |
| Preservatives, perfume | q.s. |
| Ethanol | 3.00 |
| Water | ad 100 |
| Example No. 13 | |
| Cetyldimethicone copolyol | 2.50 |
| 2-Ethylhexylmethoxycinnamate | 8.00 |
| 2,4-Bis-(4-(2-ethylhexyloxy-2-hydroxy)-phenyl-6-(4- | 2.50 |
| methoxyphenyl)-(1,3,5)-triazine | |
| Diethylhexyl butamidotriazone | 1.00 |
| 4-Methylbenzylidene camphor | 2.00 |
| Octocrylene | 2.50 |
| Phenylene-1,4-bis-(monosodium,-2-benzimidazyl-5,7- | 2.00 |
| disulfonic acid) | |
| Titanium dioxide | 2.00 |
| Zinc oxide | 1.00 |
| Dimethicone polydimethylsiloxane | 4.00 |
| Phenylmethylpolysiloxane | 25.00 |
| Octoxyglycerin | 0.30 |
| Glycerin | 7.50 |
| Glycine soy | 1.00 |
| Magnesium sulfate | 0.50 |
| Creatine | 1.50 |
| Creatinine | 0.45 |
| Retinyl palmitate | 0.01 |
| Preservatives, perfume | q.s. |
| Water | ad 100 |
| Example No. 14 | |
| PEG-30-dipolyhydroxystearate | 5.00 |
| Butylmethoxy dibenzoylmethane | 2.00 |
| Ethylhexyl triazone | 3.00 |
| Octocrylene | 4.00 |
| Phenylene-1,4-bis-(monosodium,-2-benzimidazyl-5,7- | 0.50 |
| disulfonic acid) | |
| Titanium dioxide | 1.50 |
| Zinc oxide | 2.00 |
| Paraffinum liquidum | 10.0 |
| Butyleneglycol dicaprylate/dicaprate | 2.00 |
| Dicaprylylcarbonate | 6.00 |
| Dimethicone polydimethylsiloxane | 1.00 |
| Shea butter | 3.00 |
| Octoxyglycerin | 1.00 |
| Glycine soy | 1.50 |
| Magnesium chloride | 1.00 |
| Tocopherol acetate | 0.25 |
| Creatine | 4.00 |
| Creatinine | 0.30 |
| Retinyl palmitate | 1.00 |
| Preservatives, perfume | q.s. |
| Ethanol | 1.50 |
| Water | ad 100 |
| Example No. 15 | |
| Cetyldimethicone copolyol | 4.00 |
| 2-Ethylhexyl methoxycinnamate | 5.00 |
| 2,4-Bis-(4-(2-ethylhexyloxy-2-hydroxy)-phenyl-6-(4- | 2.00 |
| methoxyphenyl)-(1,3,5)-triazine | |
| Butylmethoxy dibenzoylmethane | 1.00 |
| Ethylhexyl triazone | 4.00 |
| 4-Methylbenzylidene camphor | 4.00 |
| Diethylhexyl butamidotriazone | 2.00 |
| Phenylbenzimidazole sulfonic acid | 3.00 |
| Zinc oxide | 0.50 |
| C₁₂₋₁₅ Alkyl benzoate | 9.00 |
| Butyleneglycol dicaprylate/dicaprate | 8.00 |
| Dimethicone polydimethylsiloxane | 5.00 |
| PVP hexadecene copolymer | 0.50 |
| Glycerin | 7.50 |
| Magnesium sulfate | 0.50 |
| Creatine | 1.00 |
| Creatinine | 0.30 |
| Retinyl palmitate | 0.03 |
| Preservatives, perfume | q.s. |
| Water | ad 100 |
| Example No. 16 | |
| Polyglyceryl-2-dipolyhydroxystearate | 4.50 |
| 2-Ethylhexyl methoxycinnamate | 4.00 |
| 2,4-Bis-(4-(2-ethylhexyloxy-2-hydroxy)-phenyl-6-(4- | 2.50 |
| methoxyphenyl)-(1,3,5)-triazine | |
| Diethylhexyl butamidotriazone | 3.00 |
| Ethylhexyl triazone | |
| 4-methylbenzylidene camphor | 2.00 |
| Octocrylene | 2.50 |
| Phenylbenzimidazole sulfonic acid | 2.00 |
| Titanium dioxide | 3.00 |
| Paraffinum liquidum | 8.00 |
| Dicaprylylether | 7.00 |
| Butyleneglycol dicaprylate/dicaprate | 4.00 |
| Phenylmethylpolysiloxane | 2.00 |
| PVP hexadecene copolymer | 1.00 |
| Octoxyglycerin | 0.50 |
| Glycerin | 2.50 |
| Magnesium chloride | 0.70 |
| Tocopherol acetate | 1.00 |
| Creatine | 1.00 |
| Creatinine | 0.30 |
| Retinyl palmitate | 0.03 |
| Preservatives, perfume | q.s. |
| Ethanol | 1.00 |
| Water | ad 100 |

Examples W/O Emulsions

| | % by weight |
|---|---|
| Example No. 17 | |
| Polyglyceryl-2-dipolyhydroxystearate | 4.00 |
| Lanolin alcohol | 0.50 |
| Isohexadecane | 1.00 |
| Myristyl myristate | 0.50 |
| Vaseline | 1.00 |
| Butylmethoxy dibenzoylmethane | 0.50 |
| 4-Methylbenzylidene camphor | 1.00 |
| Butyleneglycol dicaprylate/dicaprate | 4.00 |
| Glycerin | 5.00 |
| Tocopherol acetate | 0.50 |
| Creatine | 1.00 |
| Creatinine | 0.30 |
| Retinyl palmitate | 0.03 |
| EDTA | 0.20 |
| Preservatives | q.s. |
| Perfume | q.s. |
| Water | ad 100 |
| Example No. 18 | |
| Polyglyceryl-2-dipolyhydroxystearate | 5.00 |
| Lanolin alcohol | 1.50 |
| Isohexadecane | 2.00 |
| Myristyl myristate | 1.50 |
| Vaseline | 2.00 |
| Butylmethoxy dibenzoylmethane | 1.50 |
| 4-Methylbenzylidene camphor | 3.00 |
| Butyleneglycol dicaprylate/dicaprate | 5.00 |
| Shea butter | 0.50 |
| Butylene glycol | 6.00 |
| Octoxyglycerin | 3.00 |
| Tocopherol acetate | 1.00 |
| Creatine | 2.00 |
| Creatinine | 0.50 |
| Retinylpalmitate | 3.00 |
| EDTA | 0.20 |
| Preservatives | q.s. |
| Ethanol | 3.00 |
| Perfume | q.s. |
| Water | ad 100 |
| Example No. 19 | |
| Polyglyceryl-3-diisostearate | 3.50 |
| Glycerin | 3.00 |
| Polyglyceryl-2-dipolyhydroxystearate | 3.50 |
| Creatine | 1.50 |
| Creatinine | 0.50 |
| Retinyl palmitate | 0.05 |
| Preservatives | q.s. |
| Perfume | q.s. |
| Magnesium sulfate | 0.6 |
| Isopropylstearate | 2.0 |
| Caprylyl ether | 8.0 |
| Cetearyl isononanoate | 6.0 |
| Water | ad 100 |
| Example No. 20 | |
| Triceteareth-4-phosphate | 0.80 |
| Butylated hydroxytoluene | 0.05 |
| Glyceryl lanolate | 1.70 |
| Cyclomethicone | 2.20 |
| Isopropyl palmitate | 1.00 |
| Creatine | 2.00 |
| Creatinine | 0.20 |
| Retinyl palmitate | 0.01 |
| Polyacrylic acid | 0.50 |
| Ethylenediamine tetraacetic acid | 1.00 |
| Sodium hydroxide | q.s. |
| Citric acid | 0.01 |
| Preservatives | q.s. |
| Perfume | q.s. |
| Water | ad 100 |

Examples (Gel Creams)

| | % by weight |
|---|---|
| Example No. 21 | |
| Acrylate/C10-30 alkyl acrylate crosspolymer | 0.40 |
| Polyacrylic acid | 0.20 |
| Xanthan gum | 0.10 |
| Cetyl stearyl alcohol | 3.00 |
| C₁₂₋₁₅ alkyl benzoate | 4.00 |
| Caprylic acid/capric acid triglycerides | 3.00 |
| Cyclic dimethylpolysiloxane | 5.00 |
| Dimethicone polydimethylsiloxane | 1.00 |
| Creatine | 1.50 |
| Creatinine | 1.00 |
| Retinyl palmitate | |
| Ubiquinone (Q10) | 0.50 |
| Sodium hydroxide | q.s. |
| Preservatives | q.s. |
| Perfume | q.s. |
| Water | ad 100.0 |
| pH value adjusted to 6.0 | |
| Example No. 22 | |
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C₁₂₋₁₅ Alkyl benzoate | 4.00 |
| Caprylic acid/capric acid triglycerides | 3.00 |
| Cyclic dimethylpolysiloxane | 5.00 |
| Dimethicone polydimethylsiloxane | 1.00 |
| Creatine | 1.00 |
| Creatinine | 0.30 |
| Retinyl palmitate | 0.04 |
| Glycerin | 2.00 |
| Sodium hydroxide | q.s. |
| Preservatives | q.s. |
| Perfume | q.s. |
| Water | ad 100.0 |

Examples (Hydrodispersions)

| | % by weight |
|---|---|
| Example No. 23 | |
| Polyoxyethylene(20)cetyl stearyl ether | 1.00 |
| Acrylate/C10-30 alkyl acrylate crosspolymer | 0.50 |
| 1-(4-tert-butylphenyl)-3-(4-methoxyphenyl)-1,3- | 1.00 |
| propandione | |
| Ethylhexyl triazone | 4.00 |
| 4-methylbenzylidene camphor | 4.00 |
| Phenylene-1,4-bis-(monosodium,-2-benzimidazyl-5,7- | 1.00 |
| disulfonic acid) | |
| Phenylbenzimidazole sulfonic acid | 0.50 |
| Titanium dioxide | 0.50 |
| Zinc oxide | 0.50 |
| C₁₂₋₁₅ Alkyl benzoate | 2.00 |
| Butyleneglycol dicaprylate/dicaprate | 4.00 |
| Phenylmethylpolysiloxane | 2.00 |
| PVP hexadecene copolymer | 0.50 |
| Glycerin | 3.00 |
| Tocopherol acetate | 0.50 |
| Creatine | 0.50 |
| Creatinine | 0.75 |
| Retinyl palmitate | 1.00 |
| Preservatives | q.s. |
| Ethanol | 3.00 |
| Perfume | q.s. |
| Water | ad 100.0 |
| Example No. 24 | |
| Sodium polyacrylate | 0.20 |
| Xanthan gum | 0.30 |
| 2,4-Bis-(4-(2-ethylhexyloxy-2-hydroxy)-phenyl-6-(4- | 1.50 |
| methoxyphenyl)-(1,3,5)-triazine | |
| Diethylhexyl butamidotriazone | 2.00 |
| 4-Methylbenzylidene camphor | 4.00 |
| Octocrylene | 4.00 |
| Zinc oxide | 1.00 |
| C₁₂₋₁₅ alkyl benzoate | 2.50 |
| Dicaprylyl ether | 4.00 |
| Dicaprylyl carbonate | 2.00 |
| Dimethicone polydimethylsiloxane | 0.50 |
| Shea butter | 2.00 |
| Glycerin | 6.50 |
| Creatine | 1.50 |
| Creatinine | 0.45 |
| Retinyl palmitate | 0.03 |
| Preservatives | q.s. |
| Ethanol | 2.00 |
| Perfume | q.s. |
| Water | ad 100.0 |
| Example No. 25 | |
| Cetyl alcohol | 1.00 |
| Acrylate/C10-30 alkyl acrylate crosspolymer | 0.40 |
| Xanthan gum | 0.15 |
| 1-(4-tert-butylphenyl)-3-(4-methoxyphenyl)-1,3- | 2.00 |
| propandione | |
| Ethylhexyl triazone | 3.00 |
| Octocrylene | 4.00 |
| Phenylene-1,4-bis-(monosodium,-2-benzimidazyl-5,7- | 0.50 |
| disulfonic acid) | |
| Titanium dioxide | 2.00 |
| Zinc oxide | 3.00 |
| Butyleneglycol dicaprylate/dicaprate | 2.00 |
| Dicaprylyl carbonate | 6.00 |
| Dimethicone polydimethylsiloxane | 1.00 |
| Octoxyglycerin | 1.00 |
| Glycerin | 5.00 |
| Glycine soy | 1.50 |
| Tocopherol acetate | 0.25 |
| Creatine | 0.50 |
| Creatinine | 1.00 |
| Retinyl palmitate | 0.05 |
| Preservatives | q.s. |
| Ethanol | 1.50 |
| Perfume | q.s. |
| Water | ad 100.0 |
| Example No. 26 | |
| Polyoxyethylene(20)cetyl stearyl ether | 0.5 |
| Sodium polyacrylate | 0.30 |
| Acrylate/C10-30 alkyl acrylate crosspolymer | 0.10 |
| 2-Ethylhexyl methoxycinnamate | 5.00 |
| 2,4-Bis-(4-(2-ethylhexyloxy-2-hydroxy)-phenyl-6-(4- | 2.00 |
| methoxyphenyl)-(1,3,5)-triazine | |
| Diethylhexyl butamidotriazone | 2.00 |
| Ethylhexyl triazone | 4.00 |
| Phenylbenzimidazole sulfonic acid | 3.00 |
| Titanium dioxide | 3.00 |
| Butyleneglycol dicaprylate/dicaprate | 6.00 |
| Phenylmethylpolysiloxane | 0.50 |
| PVP hexadecene copolymer | 0.50 |
| Glycerin | 5.50 |
| Creatine | 1.00 |
| Creatinine | 0.30 |
| Retinyl palmitate | 0.03 |
| Preservatives | q.s. |
| Perfume | q.s. |
| Water | ad 100.0 |
| Example No. 27 | |
| Acrylate/C10-30 alkyl acrylate crosspolymer | 0.10 |
| Xanthan gum | 0.50 |
| 2-Ethylhexyl methoxycinnamate | 8.00 |
| 2,4-Bis-(4-(2-ethylhexyloxy-2-hydroxy)-phenyl-6-(4- | 2.50 |
| methoxyphenyl)-(l,3,5)-triazine | |
| Diethylhexyl butamidotriazone | 1.00 |
| 4-Methylbenzylidene camphor | 2.00 |
| Octocrylene | 2.50 |
| Phenylene-l,4-bis-(monosodium,-2-benzimidazyl-5,7- | 2.00 |
| disulfonic acid) | |
| Titanium dioxide | 1.00 |
| Zinc oxide | 2.00 |
| Phenylmethylpolysiloxane | 2.00 |
| PVP hexadecene copolymer | 1.00 |
| Octoxyglycerin | 0.50 |
| Glycerin | 1.50 |
| Tocopherol acetate | 1.00 |
| Creatine | 2.00 |
| Retinyl palmitate | 0.25 |
| Preservatives | q.s. |
| Ethanol | 1.00 |
| Perfume | q.s. |
| Water | ad 100.0 |
| Example No. 28 | |
| Polyoxyethylene(20)cetyl stearyl ether | 1.00 |
| Acrylate/C10-30 alkyl acrylate crosspolymer | 0.50 |
| 1-(4-tert-Butylphenyl)-3-(4-methoxyphenyl)-1,3- | 1.00 |
| propandione | |
| Ethylhexyl triazone | 4.00 |
| 4-Methylbenzylidene camphor | 4.00 |
| Phenylene-1,4-bis-(monosodium,-2-benzimidazyl-5,7- | 1.00 |
| disulfonic acid) | |
| Phenylbenzimidazole sulfonic acid | 0.50 |
| Titanium dioxide | 0.50 |
| Zinc oxide | 0.50 |
| C₁₂₋₁₅ Alkyl benzoate | 2.00 |
| Butyleneglycol dicaprylate/dicaprate | 4.00 |
| Phenylmethylpolysiloxane | 2.00 |
| PVP hexadecene copolymer | 0.50 |
| Glycerin | 3.00 |
| Tocopherol acetate | 0.50 |
| Creatine | 1.00 |
| Creatinine | 0.30 |
| Retinyl palmitate | 0.04 |
| Preservatives | q.s. |
| Ethanol | 3.00 |
| Perfume | q.s. |
| Water | ad 100.0 |
| Example No. 29 | |
| Sodium polyacrylate | 0.20 |
| Xanthan gum | 0.30 |
| 2,4-Bis-(4-(2-ethylhexyloxy-2-hydroxy)-phenyl-6-(4- | 1.50 |
| methoxyphenyl)-(1,3,5)-triazine | |
| Diethylhexyl butamidotriazone | 2.00 |
| 4-Methylbenzylidene camphor | 4.00 |
| Octocrylene | 4.00 |
| Zinc oxide | 1.00 |
| C₁₂₋₁₅ Alkyl benzoate | 2.50 |
| Dicaprylyl ether | 4.00 |
| Dicaprylyl carbonate | 2.00 |
| Dimethicone polydimethylsiloxane | 0.50 |
| Shea butter | 2.00 |
| Glycerin | 6.50 |
| Creatine | 1.50 |
| Creatinine | 0.45 |
| Retinyl palmitate | 0.30 |
| Preservatives | q.s. |
| Ethanol | 2.00 |
| Perfume | q.s. |
| Water | ad 100.0 |
| Example No. 30 | |
| Cetyl alcohol | 1.00 |
| Acrylate/C10-30 alkyl acrylate crosspolymer | 0.40 |
| Xanthan gum | 0.15 |
| 1-(4-tert-Butylphenyl)-3-(4-methoxyphenyl)-1,3- | 2.00 |
| propandione | |
| Ethylhexyl triazone | 3.00 |
| Octocrylene | 4.00 |
| Phenylene-1,4-bis-(monosodium,-2-benzimidazyl-5,7- | 0.50 |
| disulfonic acid) | |
| Titanium dioxide | 2.00 |
| Zinc oxide | 3.00 |
| Butyleneglycol dicaprylate/dicaprate | 2.00 |
| Dicaprylyl carbonate | 6.00 |
| Dimethicone polydimethylsiloxane | 1.00 |
| Octoxyglycerin | 1.00 |
| Glycerin | 5.00 |
| Glycine soy | 1.50 |
| Tocopherol acetate | 0.25 |
| Creatine | 0.50 |
| Creatinine | 0.20 |
| Retinyl palmitate | 0.025 |
| Preservatives | q.s. |
| Ethanol | 1.50 |
| Perfume | q.s. |
| Water | ad 100.0 |
| Example No. 31 | |
| Polyoxyethylene(20)cetyl stearyl ether | 0.5 |
| Sodium polyacrylate | 0.30 |
| Acrylate/C10-30 alkyl acrylate crosspolymer | 0.10 |
| 2-Ethylhexyl methoxycinnamate | 5.00 |
| 2,4-Bis-(4-(2-ethylhexyloxy-2-hydroxy)-phenyl-6-(4- | 2.00 |
| methoxyphenyl)-(1,3,5)-triazine | |
| Diethylhexyl butamidotriazone | 2.00 |
| Ethylhexyl triazone | 4.00 |
| Phenylbenzimidazole sulfonic acid | 3.00 |
| Titanium dioxide | 3.00 |
| Butyleneglycol dicaprylate/dicaprate | 6.00 |
| Phenylmethylpolysiloxane | 0.50 |
| PVP hexadecene copolymer | 0.50 |
| Glycerin | 5.50 |
| Creatine | 1.00 |
| Creatinine | 0.50 |
| Retinyl palmitate | 0.02 |
| Preservatives | q.s. |
| Perfume | q.s. |
| Water | ad 100.0 |
| Example No. 32 | |
| Acrylate/C10-30 alkyl acrylate crosspolymer | 0.10 |
| Xanthan gum | 0.50 |
| 2-Ethylhexyl methoxycinnamate | 8.00 |
| 2,4-Bis-(4-(2-ethylhexyloxy-2-hydroxy)-phenyl-6-(4- | 2.50 |
| methoxyphenyl)-(1,3,5)-triazine | |
| Diethylhexyl butamidotriazone | 1.00 |
| 4-Methylbenzylidene camphor | 2.00 |
| Octocrylene | 2.50 |
| Phenylene-1,4-bis-(monosodium,-2-benzimidazyl-5,7- | 2.00 |
| disulfonic acid) | |
| Titanium dioxide | 1.00 |
| Zinc oxide | 2.00 |
| Phenylmethylpolysiloxane | 2.00 |
| PVP hexadecene copolymer | 1.00 |
| Octoxyglycerin | 0.50 |
| Glycerin | 1.50 |
| Tocopherol acetate | 1.00 |
| Creatine | 2.00 |
| Creatinine | 0.50 |
| Retinyl palmitate | 0.05 |
| Preservatives | q.s. |
| Ethanol | 1.00 |
| Perfume | q.s. |
| Water | ad 100.0 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. An active substance combination comprising
(b) at least one of creatine, creatinine, a creatine derivative and a creatinine derivative, and
(b) at least one retinoid.

2. The active substance combination of claim 1, wherein a weight ratio of creatinine to creatine is from about 10:1 to about 1:10.

3. The active substance combination of claim 2, wherein the weight ratio is from about 4:1 to about 3:7.

4. The active substance combination of claim 2, wherein the weight ratio is from about 2:1 to about 1:2.

5. The active substance combination of claim 1, wherein the at least one retinoid comprises at least one of retinol and retinyl palmitate.

6. The active substance combination of claim 1, wherein the at least one retinoid comprises retinyl palmitate.

7. The active substance combination of claim 1, wherein the at least one retinoid comprises retinol.

8. A cosmetic or dermatological preparation which comprises an effective amount of the active substance combination of claim 1.

9. The preparation of claim 8, wherein the preparation comprises from about 0.0005% to about 50% by weight of the active substance combination.

10. The preparation of claim 9, wherein the preparation comprises from about 0.01% to about 20% by weight of the active substance combination.

11. The preparation of claim 10, wherein the preparation comprises from about 0.02% to about 10% by weight of the active substance combination.

12. The preparation of claim 11, wherein the preparation comprises from about 0.02% to about 5% by weight of the active substance combination.

13. The preparation of claim 12, wherein the preparation comprises from about 0.5% to about 3% by weight of the active substance combination.

14. The preparation of claim 8, wherein the preparation comprises from about 0.001% to about 10% by weight of at least one of creatine and a creatine derivative.

15. The preparation of claim 14, wherein the preparation comprises from about 0.01% to about 1% by weight of at least one of creatine and a creatine derivative.

16. The preparation of claim 8, wherein the preparation comprises from about 0.001% to about 10% by weight of at least one of creatinine and a creatinine derivative.

17. The preparation of claim 15, wherein the preparation comprises from about 0.01% to about 1% by weight of at least one of creatinine and a creatinine derivative.

18. The preparation of claim 8, wherein the preparation comprises from about 0.001% to about 10% by weight of the at least one retinoid.

19. The preparation of claim 12, wherein the preparation comprises from about 0.01% to about 1% by weight of the at least one retinoid.

20. The preparation of claim 19, wherein the at least one retinoid comprises at least one of retinol and retinyl palmitate.

21. The preparation of claim 8, wherein the preparation further comprises from about 0.001% to about 30% by weight of glycerin.

22. The preparation of claim 9, wherein the preparation further comprises from about 0.01% to about 15% by weight of glycerin.

23. The preparation of claim 10, wherein the preparation further comprises from about 0.01% to about 15% by weight of glycerin.

24. The preparation of claim 11, wherein the preparation further comprises from about 1% to about 7% by weight of glycerin.

25. A cosmetic or dermatological preparation which comprises an effective amount of the active substance combination of claim 2.

26. A cosmetic or dermatological preparation which comprises an effective amount of the active substance combination of claim 3.

27. A cosmetic or dermatological preparation which comprises an effective amount of the active substance combination of claim 4.

28. A cosmetic or dermatological preparation which comprises an effective amount of the active substance combination of claim 5.

29. A cosmetic or dermatological preparation which comprises from about 0.01% to about 20% by weight of an active substance combination comprising
(a) at least one of creatine, creatinine, a creatine derivative and a creatinine derivative, and
(b) at least one retinoid.

30. The preparation of claim 29, wherein a weight ratio of creatinine to creatine in the active substance combination is from about 4:1 to about 3:7.

31. The preparation of claim 30, wherein the weight ratio is from about 2:1 to about 1:2.

32. The preparation of claim 29, wherein the preparation comprises from about 0.02% to about 10% by weight of the active substance combination.

33. The preparation of claim 30, wherein the preparation comprises from about 0.5% to about 3% by weight of the active substance combination.

34. The preparation of claim 32, wherein the preparation comprises from about 0.01% to about 1% by weight of at least one of creatine and a creatine derivative.

35. The preparation of claim 32, wherein the preparation comprises from about 0.01% to about 1% by weight of at least one of creatinine and a creatinine derivative.

36. The preparation of claim 31, wherein the preparation further comprises from about 0.01% to about 15% by weight of glycerin.

37. The preparation of claim 30, wherein the preparation further comprises from about 0.01% to about 15% by weight of glycerin.

38. The preparation of claim 32, wherein the preparation further comprises from about 1% to about 7% by weight of glycerin.

39. The preparation of claim 32, wherein the preparation comprises from about 0.001% to about 10% by weight of the at least one retinoid.

40. The preparation of claim 30, wherein the preparation comprises from about 0.01% to about 1% by weight of the at least one retinoid.

41. The preparation of claim 40, wherein the at least one retinoid comprises at least one of retinol and retinyl palmitate.

42. An O/W cream which comprises the active substance combination of claim 1.

43. An O/W emulsion which comprises the active substance combination of claim 1.

44. A W/O emulsion which comprises the active substance combination of claim 1.

45. A gel cream which comprises the active substance combination of claim 1.

46. A hydrodispersion which comprises the active substance combination of claim 1.

47. A method for the prophylaxis or treatment of UV or ozone-induced skin damage, wherein the method comprises applying to at least a part of the skin the preparation of claim 8.

48. A method for the prophylaxis or treatment of inflammatory and degenerative skin, wherein the method comprises applying to at least a part of the skin the preparation of claim 8.
